(19) United States
(12) Patent Application Publication
(10) Pub. No.: US 2008/0299592 A1
(43) Pub. Date: Dec. 4, 2008

(54) Red-Shifted Luciferase

(76) Inventor: Stephen C. Miller, Cambridge, MA (US)

Correspondence Address: FISH & RICHARDSON PC, P.O. BOX 1022, MINNEAPOLIS, MN, 55440-1022, US

(21) Appl. No.: 12/040,797

(22) Filed: Feb. 29, 2008

Related U.S. Application Data
(60) Provisional application No. 60/904,582, filed on Mar. 2, 2007.

(51) Int. Cl. C12Q 1/66; C07K 7/00; C12N 9/02; C12N 15/11; C07F 9/66; C07F 9/92; C12N 5/06; C12N 15/00; A01K 67/027

(52) U.S. Cl. 435/8; 530/300; 435/189; 536/23.2; 435/325; 435/320.1; 800/13; 800/18; 544/64

(57) ABSTRACT

The compositions described herein shift the light output of luciferases to the near-IR by resonance energy transfer to a targetable near-IR fluorophore.

### CLAIM OF PRIORITY

This application claims the benefit under 35 USC § 119(e) of U.S. Provisional Patent Application Ser. No. 60/904,582, filed on Mar. 2, 2007, the entire contents of which are incorporated herein by reference.

### TECHNICAL FIELD

This invention relates to luciferase constructs with red-shifted emissions, and methods of using them.

### BACKGROUND

Modern genomics and high-throughput screening technologies have allowed the identification of a large number of potential therapeutic targets and drugs for the treatment of diseases such as cancer. However, therapeutic strategies that show great promise in cultured cells often fail in whole organisms. Validation of target proteins and therapeutic compounds in live animals is thus a fundamentally important step in the development of effective therapies.

Imaging of tumor cell growth and metastasis in live animals gives a much more detailed and accurate picture of overall disease progression and, for example, response to drug intervention, than can be obtained from cultured cells. In particular, bioluminescent imaging (BLI) with firefly luciferase has gained widespread acceptance as a powerful, inexpensive, and non-invasive method to monitor gene expression, enzymatic activity, protein-protein interactions and protein degradation in the context of the whole organism (Massoud and Gambhir, Genes Dev, 2003. 17(5):545-80). Relative to other imaging modalities such as PET and MRI, bioluminescence imaging has the advantages of low cost, speed, sensitivity, high throughput and ease of use by non-specialists (Shah et al., Gene Ther, 2004. 11(15):1175-87). These advantages make BLI the method of choice for rapidly assessing disease progression and response to potential therapeutics in mice.

The major limitations of BLI are the poor penetration of visible light through tissue and the lower 3D spatial resolution relative to more specialized imaging techniques. Illuminated tissue is most transparent to near-IR light (650-900 nm), where autofluorescence is minimal, Rayleigh scattering is greatly decreased, and the absorption of visible light by hemoglobin is at its lowest (Weissleder and Ntziachristos, Nat. Med., 2003, 9(1):123-8).

### SUMMARY

The compositions described herein shift the light output of luciferase to the near-IR by resonance energy transfer to a targetable near-IR acceptor fluorophore. The efficiency of the energy transfer can be further optimized by varying the acceptor fluorophore, varying the orientation of the acceptor fluorophore and the luciferin, and adjusting, e.g., decreasing, the distance between the luciferin chromophore and the acceptor fluorophore. The compositions described herein also include targetable, cell-permeable small molecule near-IR fluorophores.

In one aspect, the invention features isolated nucleic acid molecules including a sequence of nucleotides that encode a modified luciferase polypeptide, including a luciferase polypeptide (e.g., luciferase from a firefly, a Renilla, a click beetle, a bacterium (e.g., luxAB), or a railroad worm) and at least one tetracysteine tag comprising the amino acid sequence CCXXCC (SEQ ID NO:11). Alternatively, in place of or in addition to the tetracysteine tag, the nucleic acid molecules can include a sequence encoding a HaloTag™ protein.

In some embodiments, these isolated nucleic acid molecules are cloned in frame with a second nucleic acid molecule including a second sequence of nucleotides encoding a preselected protein, and optionally a third sequence of nucleotides encoding a linker between the sequence of nucleotides encoding the modified luciferase and the second sequence of nucleotides encoding the preselected protein.

In some embodiments, the isolated nucleic acid molecules described herein are operably linked to a preselected regulatory sequence, enhancer sequence, silencer sequence, or promoter.

Also provided herein are host cells including the nucleic acid molecules described herein; vectors including the nucleic acid molecules described herein; and host cells including those vectors.

In another aspect, the invention provides isolated polypeptides including a luciferase polypeptide and at least one tetracysteine tag comprising the sequence CCXXCC (SEQ ID NO:11), e.g., inserted at the N terminus, the C terminus, and/or internally into the luciferase sequence. In addition, the invention provides isolated polypeptides including a luciferase polypeptide fused in frame with at least one HaloTag™ protein at one or more of the N terminus and the C terminus.

In some embodiments, these isolated polypeptides also include a protein of interest fused in frame with the luciferase and tetracysteine tag or HaloTag™ protein.

In a further aspect, the invention features transgenic non-human mammals, e.g., mice, the nucleated cells of which include a transgene encoding an isolated polypeptide including a modified luciferase polypeptide as described herein, wherein the polypeptide is expressed in at least some of the cells. The invention also features transgenic non-human mammals, e.g., mice, whose genome is heterozygous for a transgene encoding an isolated polypeptide including a modified luciferase polypeptide as described herein, wherein the polypeptide is expressed in at least some of the cells of the mouse.

In an additional aspect, the invention provides methods for imaging gene expression in a living cell. The methods include providing a cell expressing a modified luciferase polypeptide as described herein that includes a tetracysteine tag; contacting the cell with luciferin; contacting the cell with a near-infrared (NIR) acceptor dye that binds to the polypeptide, e.g., a bis-arsenical dye that fluoresces above 600 nm and undergoes intramolecular biofluorescence resonance energy transfer (BRET) with the modified luciferase polypeptide; and detecting NIR emission from the NIR acceptor dye.

In yet another aspect, the invention provides methods for imaging gene expression in a living cell. The methods include providing a cell expressing a modified luciferin polypeptide as described herein that contains a HaloTag™ protein; contacting the cell with luciferin; contacting the cell with a near-infrared (NIR) acceptor dye that binds to the polypeptide, e.g., a chloroalkyl-tethered fluorophore dye that fluoresces above 600 nm and undergoes intramolecular biofluorescence resonance energy transfer (BRET) with the modified luciferase polypeptide; and detecting NIR emission from the NIR acceptor dye.

In another aspect, the invention features compounds that include cations of Structure (I), which is shown below.

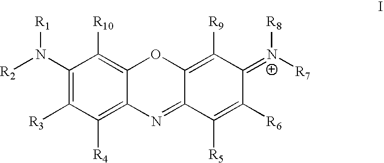

In such compounds, each R₄ and R₅ or each R₉ and R₁₀ is an arsenic-containing moiety or an antimony-containing moiety.

When R₄ and R₅ are each an arsenic-containing moiety or an antimony-containing moiety, R₃, R₆, R₉ and R₁₀ are each independently H, F, Cl, Br, I, OH, or a first moiety that includes up to 12 carbon atoms, R₁, R₂, R₇ and R₈ are each independently H or a second moiety that includes up to 12 carbon atoms, and R₁, R₂, R₃ and R₁₀ and/or R₆, R₇, R₈ and R₉ together with one or more of its immediate neighbors can define one or more ring systems, each including up to 14 carbon atoms.

When R₉ and R₁₀ are each an arsenic-containing moiety or an antimony-containing moiety, R₁ and R₈ are each H, R₂ and R₇ are each independently H or together with its immediate respective neighbor defines one or more ring systems, each including up to 14 carbon atoms, R₃, R₄, R₅ and R₆ are each independently H, F, Cl, Br, I, OH, or third moiety that includes up to 12 carbon atoms, and R₃ and R₄ and/or R₅ and R₆ together with one or more of its immediate neighbors may define one or more ring systems, each including up to 14 carbon atoms.

In some embodiments, R₉ and R₁₀ are each an arsenic-containing moiety or an antimony-containing moiety, R₄ and R₅ are each H, and R₂ and R₃ and R₆ and R₇ together define one or more ring systems, each including up to 14 carbon atoms. For example, each ring system can be a 6-membered ring system.

For example, the cations can be represented by Structure (6b) or (6d), which are shown below.

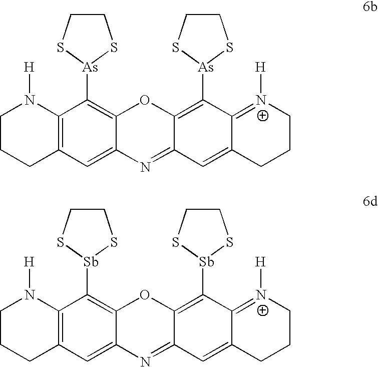

In some embodiments, R₄ and R₅ are each an arsenic-containing moiety or an antimony-containing moiety. For example, the cations can be represented by Structure (6c) or (6c′), which are shown below.

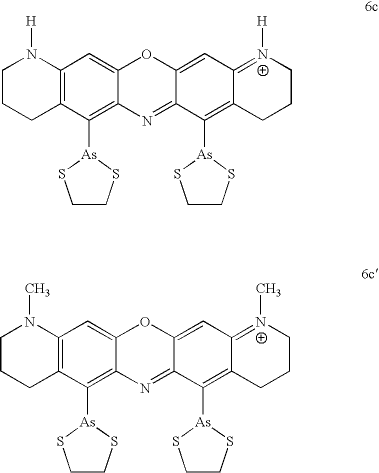

In another aspect, the invention features compounds that include cations of Structure (VI), which is shown below.

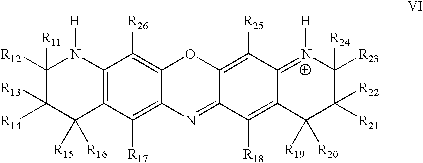

In such cations, each R₁₇ and R₁₈ or each R₂₅ and R₂₆ is an arsenic-containing moiety, a mercury-containing moiety, or an antimony-containing moiety.

When R₁₇ and R₁₈ are each an arsenic-containing moiety or an antimony-containing moiety, R₁₁, R₁₂, R₁₃, R₁₄, R₁₅, R₁₆, R₁₉, R₂₀, R₂₁, R₂₂, R₂₃, R₂₄, R₂₅ and R₂₆ are each independently H, or a moiety that includes up to 8 carbon atoms,

When R₂₅ and R₂₆ are each an arsenic-containing moiety or an antimony-containing moiety, R₁₁, R₁₂, R₁₃, R₁₄, R₁₅, R₁₆, R₁₇, R₁₈, R₁₉, R₂₀, R₂₁, R₂₂, R₂₃ and R₂₄ are each independently H, or a moiety that includes up to 8 carbon atoms.

In some embodiments, wherein R₁₇ and R₁₈ are each an arsenic-containing moiety or an antimony-containing moiety and wherein R₁₁, R₁₂, R₁₃, R₁₄, R₁₅, R₁₆, R₁₉, R₂₀, R₂₁, R₂₂, R₂₃, R₂₄, R₂₅ and R₂₆ are each H.

In other embodiments, R₂₅ and R₂₆ are each an arsenic-containing moiety or an antimony-containing moiety and wherein R₁₁, R₁₂, R₁₃, R₁₄, R₁₅, R₁₆, R₁₇, R₁₈, R₁₉, R₂₀, R₂₁, R₂₂, R₂₃ and R₂₄ are each H.

The compounds described herein can further include, e.g., ClO₄⁻, BF₄⁻ or PF₆⁻ as a counterion.

In another aspect, the invention features conjugates of any compound described herein and a peptide, a polypeptide or a protein.

In another aspect, the invention features compositions that include any compound and/or conjugate described herein.

The invention provides several advantages. Near-IR light emission by the red-shifted luciferases described herein would allow optical imaging, e.g., of reporter gene expression, in living subjects with at least an order of magnitude greater sensitivity than is currently available with wild-type firefly luciferase. This allows more rapid image acquisition, imaging of smaller numbers of cells, and improved imaging of tumors in organs that are located deeper in the body cavity. Accelerating the rate of data acquisition and improving the detection limits of bioluminescent imaging (BLI) both broadens the scope of what can be imaged using BLI, and allows many more subjects, e.g., experimental animals, to be imaged per day. Furthermore, this approach allows subsequent fluorescence microscopy imaging of individual cells excised from the animal, providing verification of the cellular signal source as well as allowing more detailed subcellular localization of the reporter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. It is not an admission that any of the information provided herein is prior art or relevant to the presently claimed inventions, or that any publication specifically or implicitly referenced is prior art.

Other features and advantages of the invention will be apparent from the following detailed description and figures, and from the claims.

### DESCRIPTION OF DRAWINGS

FIGS. 1A-B are schematic illustrations of two strategies for targeting fluorophores to a tagged protein. 1A, a method using a fusion protein with a linker. 1B, a method using a peptide tag.

FIG. 2 illustrates the structures of known blue (CHoXAsH), green (FlAsH), and red (ReAsH and BArNile) bis-arsenical dyes.

FIGS. 3A-B are schematic illustrations of firefly luciferase emission (3A) that is shifted to the NIR by BRET to an acceptor fluorophore (3B).

FIG. 3C are spectra illustrating a shift in tetracysteine-tagged luciferase emission upon binding of the red bis-arsenical dye ReAsH.

FIG. 4 is a schematic illustration of the commercially-available chloroalkyl-tethered tetramethylrhodamine (555 nm/580 nm excite/emit).

FIG. 5 is a schematic illustration of the structure of bis-arsenical tetramethylrhodamine, which is non-fluorescent, ostensibly due to steric hindrance between the dimethylamino groups and the arsenic-EDT moiety.

FIGS. 6A-C illustrate structures of bis-arsenical dyes, including canonical bis-arsenical versions of Oxazine 170 (6A), AB2 (6B) and alternate bis-arsenical AB2 (6C).

FIGS. 7-10 are generalized structures of cations of targeting dyes.

FIGS. 11 and 12 are exemplary structures of cations of targeting dyes or precursors to targeting dyes.

FIGS. 13-16 are reaction schemes for making exemplary targeting dyes and precursors.

### DETAILED DESCRIPTION

Fluorescent imaging of tissues in the visible region of the spectrum is presently limited due to cellular autofluorescence and the high loss of both excitation and emission light to hemoglobin absorbance and Rayleigh scattering. Living tissue is most transparent to light in the near-IR range (650-900 nm). However, there are no known useful near-IR excitable fluorescent proteins. The most red-shifted fluorescent protein known is mPlum, which is maximally excited in the red (590 nm) and emits, albeit dimly, on the edge of the near-IR (649 nm) (Wang et al., Proc. Natl. Acad. Sci. USA, 2004, 101:16745-49). However, the need for excitation in the visible region combined with the very low quantum yield and extinction coefficient of this protein severely limit its utility for imaging in live animals.

Luciferases do not require excitation light and do not suffer from background autofluorescence. However, their light emission is typically also in the visible range. The blue-green emissions from Renilla luciferase (475 nm) and bacterial luciferase (495 nm) are greatly attenuated in living tissue and are not generally suitable for deep imaging. On the other hand, firefly luciferase emits maximally at 560 nm, with a broad spectrum that has a small component above 600 nm (Contag and Bachmann, Annu. Rev. Biomed. Eng., 2002, 4:235-620. After imaging through ˜1 mm of mouse tissue, the spectrum of firefly luciferase is almost completely attenuated below 600 nm (Rice et al., Journal of Biomedical Optics, 2001, 6:432-440). Due to the extremely low background, sufficient light can thus be detected to allow imaging. However, the rapid depth-based signal attenuation places a serious limitation on which organs can be effectively imaged and the minimum number of luciferase-expressing cells that can be detected.

It is instructive to compare the signal attenuation at a tissue depth of 1 cm as a function of wavelength. At 550 nm, the attenuation is 10⁻¹⁰; at 590 nm, 10⁻⁴; and 10⁻² at 650 nm (Rice et al., Journal of Biomedical Optics, 2001, 6:432-440). Thus, substantial improvement in signal would be achieved by shifting the emission wavelength to >650 nm while maintaining the high photon output of firefly luciferase (Weissleder and Ntziachristos, Nat. Med., 2003, 9:123-128). As a consequence, the ability to image smaller numbers of luciferase-expressing cells and cells that are located more deeply under the skin will improve. This is of particular importance for the detection of, e.g., infections, small tumors, early metastasis events, and cancers or infections of deep organs such as the lung or the liver. Furthermore, the increase in signal will allow more rapid image acquisition, and further improve the overall throughput of BLI.

No natural luciferase has been previously discovered or engineered to have maximal emission in the near-IR. The so-called beetle luciferases (firefly (nucleotides 76-1728 of GenBank Acc. No. X65323.2 (Promega plasmid pGL2), polypeptide is at GenBank Acc. No. CAA46419.1; additional plasmids include Promega's pGL3 (U47295.2) and pGL4 (DQ904462.1) vectors), click beetle (Pyrophorus mellifluous, GenBank Acc. No. AF545853.1 (mRNA) and AAQ19141.1 (protein), e.g., pCBR-Control plasmid AY258592.1 (nucleotides 280-1908)), and railroad worm luciferase (Phrixothrix hirtus, GenBank Acc. No. AF139645 (mRNA) and AAD34543.1 (protein)) emit light of the longest wavelengths. Railroad worm luciferase and some firefly and click beetle luciferase mutants emit at wavelengths up to 620 nm (Viviani et al., Biochemistry, 1999, 38:8271-79), albeit with a significant loss in intensity relative to wild-type firefly luciferase (560 nm) (Viviani et al., Photochem. Photobiol., 2002, 76:538-44. These beetle luciferases all use the same D-luciferin substrate, and their emission properties are thus inherently limited by the oxyluciferin chromophore. As a result, any luciferase that uses this substrate—whether discovered or engineered by mutagenesis—will not efficiently emit light in the near-IR.

### Shifting Luciferase Emission to the Near-IR: Intramolecular Bioluminescence Resonance Energy Transfer (BRET)

To enable bright luciferase emission at wavelengths>600 nm, e.g., over 650 nm, the methods and compositions described herein employ intramolecular bioluminescence resonance energy transfer (BRET) (Xu et al., Proc. Natl. Acad. Sci. USA, 1999, 96:151-156) to a targeted near-IR acceptor fluorophore, i.e., a fluorophore that is bound to the luciferase. Unbound fluorophore is generally not excited, and thus will not give rise to background signal or phototoxicity. Shifting the luciferase output to the near-IR greatly improves the tissue penetration, e.g., allowing imaging of tissues, structures and cells that are about, for example, 1 cm from the surface. An additional benefit of this strategy is the ability to image the tagged luciferase by fluorescence microscopy, allowing verification of reporter gene localization in cases where spatial resolution of the luciferase in the whole animal is limiting.

Intermolecular BRET (i.e., BRET between two molecules that are not covalently attached) between the blue-emitting Renilla luciferase and GFP or its variants has been widely used to detect protein-protein interactions in living cells (Xu et al., Methods Enzymol., 2003, 360:289-301). On the other hand, intermolecular BRET from Photinus pyralis (firefly) luciferase to an acceptor chromophore has recently been reported by the Nagamune group (Yamakawa et al., J. Biosci. Bioeng., 2002, 93:537-542; Arai et al., J. Biosci. Bioeng., 2002, 94:362-364). Nagamune et al. found that an anti-GST antibody could mediate weak BRET between GST-tagged firefly luciferase and a protein G-tagged red fluorescent protein, DsRed (Arai et al., J. Biosci. Bioeng., 2002, 94:362-364). Similarly, they observed BRET between GST-myc-luciferase and a Cy3-labeled anti-myc antibody (Yamakawa et al., J. Biosci. Bioeng., 2002, 93:537-542). While this work has demonstrated that intermolecular BRET from firefly luciferase to an acceptor fluorophore is possible, the observed energy transfer was weak, and the approach is not suitable for live cells.

Intramolecular BRET between Renilla luciferase and GFP has also been shown to cause a shift in the emission, albeit with modest efficiency (Wang et al., Mol. Genet. Genomics, 2002, 268:160-168). In addition, the first example of intramolecular BRET was recently reported between Renilla luciferase and near-IR-emitting quantum dots (So et al., Nat Biotechnol, 2006. 24(3):339-43). This work demonstrates the advantages of the BRET approach over direct fluorescence excitation, even in the near-IR. However, this method is predicated on the in vitro formation of Renilla luciferase-quantum dot complexes, and does not allow targeting of the quantum dot to a genetically-encoded luciferase. Furthermore, the quantum dots used in So et al. are not intrinsically cell-permeable, and their large size (>20 nm) restricts their ability to pass through the blood vessel wall into organs and tumors as well as their ability to be cleared from the body by the kidneys (Zhao et al., J. Biomed. Opt., 2005, 10:41210). Therefore, methods using quantum dots as described in So et al. are not suitable for use in imaging methods detecting intracellular events, subcellular localization, or intracellular function in living cells, tissues, or animals.

In contrast, the targeted small-molecule near-IR fluorophores (sNIRFs) described herein can freely access intracellular locations, and will allow the application of BRET to the imaging of gene expression, intracellular protein-protein interactions and protein degradation using existing genetically-encoded bioluminescent reporter strategies.

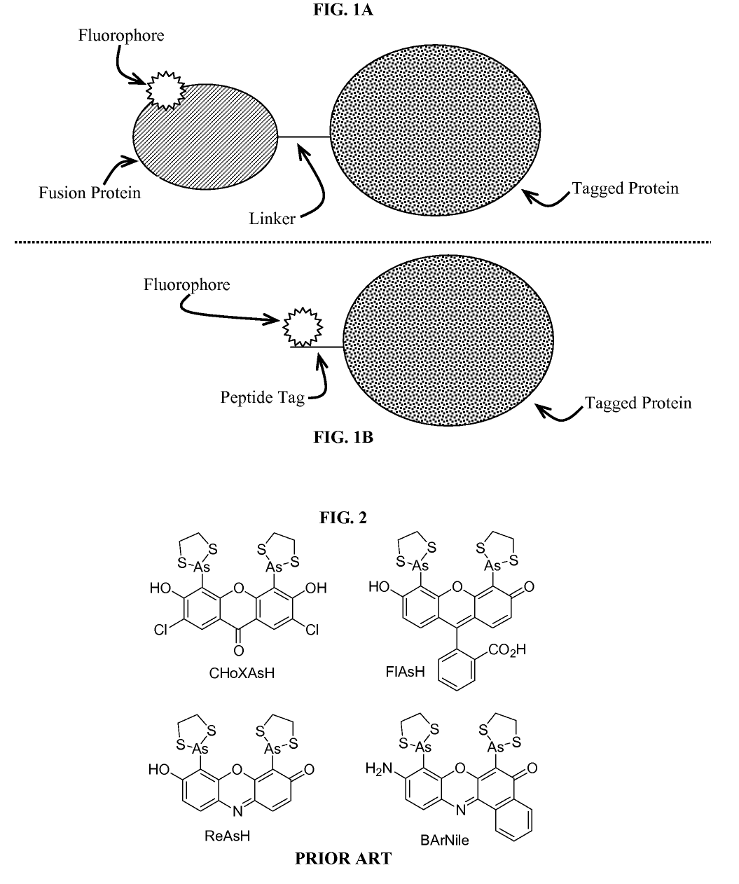

As illustrated in FIGS. 1A and 1B, two main strategies are described herein for red-shifting the emission spectra of luciferase, (1) using tetracysteine-tagged luciferase in combination with a bis-arsenical sNIRF, or (2) using a HaloTag™ protein-luciferase fusion protein in combination with a chloroalkyl-tethered fluorophore. However, other potentially suitable strategies for targeting the luciferase to a near-IR acceptor fluorophore are known to those skilled in the art, and include hexahistidine peptide tags, and fusion proteins incorporating alkyl-guanosine transferase (AGT), dihydrofolate reductase (DHFR), or FKBP.

Tetracysteine-Tagged Luciferase

The recognition of tetracysteine peptides by bis-arsenicals is a useful tool for in vivo imaging (Griffin et al., Science, 1998, 281:269-272; Adams et al., J. Am. Chem. Soc., 2002, 124:6063-76). Bis-arsenicals are cell-permeable and bind tightly to proteins containing the tetracysteine tag (i.e., CCXXCC (SEQ ID NO:11), wherein X is any amino acid, e.g., CCPGCC (SEQ ID NO:12)). Included herein are modified luciferases that include one or more tetracysteine tags, e.g., linked in tandem at the N or C terminus of the protein, one or more at each terminus, and/or one or more inserted internally into the sequence of the luciferase. For example, residues 35-40 of firefly luciferase contain a beta-bend sequence (LVPGTI (SEQ ID NO:13)) which could be replaced with CCPGCC (SEQ ID NO:12).

Methods for making tetracysteine-tagged proteins are described herein and known in the art, see, e.g., U.S. Pat. App. Pub. No. 2005/0176065 to Hanson et al. This tag is small and unlikely to perturb protein folding or cellular function. Most importantly, the close proximity of the bound fluorophore to the expressed protein is optimal for FRET and BRET applications (FIG. 1). The bis-arsenical/tetracysteine-tag technology (along with bis-antimony technology) has been successfully applied to the design of targetable, cell-permeable blue (CHoXAsH), green (FlAsH), and red (ReAsH) fluors (structures shown in FIG. 2), which do not emit in the NIR range (Adams et al., J. Am. Chem. Soc., 2002, 124:6063-76). Novel bis-arsenical near-IR fluorophores are described herein, along with related compounds.

HaloTag™ Protein-Luciferase Fusion Proteins

Among the various fusion proteins that are capable of specifically binding a small molecule covalently or with very high affinity, HaloTag™ proteins stand out for the simplicity, small size, and orthogonality of the chloroalkyl binding domain (Los et al., Journal of Neurochemistry, 2005, 94:15). See also International Application Publication No. WO 2006/093529 for a description of HaloTag™ proteins. This haloalkane dehydrohalogenase mutant forms a covalent attachment to chloroalkane-tethered small molecules, which are otherwise chemically inert. The chloroalkyl-tethered small molecule fluorophores (see, e.g., FIG. 4) are cell-permeable and are similarly expected to distribute throughout the body, and are likely to suffer less from the thiol-binding background of bis-arsenicals, and may prove to have lower toxicity, as they exhibit lower overall non-specific protein binding. Plasmids for creating HaloTag™ protein fusions are known in the art, e.g., Cloning vector pFC8A, (GenBank Acc. No. DQ137254.1) is available from Promega. A HaloTag™ protein is encoded by nucleotides 1501-2379 of the sequence shown at DQ137254.1.

The primary shortcoming of HaloTag™ protein fusions, or any fusion protein strategy, is the size of the protein and the inherent limitation on the proximity of the attached fluorophore to the luciferin binding pocket. HaloTag™ proteins are a much larger targeting sequence than the tetracysteine tag (a 33 kD protein, rather than a short peptide), and HaloTag™ protein fusions with luciferase will likely result in a lower BRET efficiency due to the larger distance between the donor and acceptor (FIG. 1).

It is expected that a C-terminal fusion of luciferase to HaloTag™ proteins would place the acceptor fluorophore about 7-7.5 nm away, without considering the tether between a HaloTag™ protein and luciferase. Linkers or tethers have been recommended to allow proper folding of the fused proteins. The recommended length is about 15-21 amino acids, not containing prolines, charged amino acids, or amino acids with bulky side chains. For example, a linker of about 17, 18, or 19 amino acids in length can be used. Depending on the structure of this tether, a HaloTag™ protein-luciferase fusion may place the fluorophore at a position too remote to engage in energy transfer, or at a distance close to the Forster radius. Thus, this strategy may suffer from a loss of BRET efficiency relative to the tetracysteine-tag approach we have demonstrated (see FIG. 1). However, the ease of appending the chloroalkyl group to any fluorophore allows this limitation to be mitigated to some degree by utilizing an acceptor fluorophore with a very large extinction coefficient at the luciferase emission wavelength. The fluorophore could thus be chosen to have the greatest possible spectral overlap integral with luciferase, and thus the largest possible Forster radius. For example, sulforhodamine 101, oxazines such as MR121, carbopyronins such as ATTO647N, and any of a number of cyanine dyes (e.g., Cy5™, Alexa™ 647) could be used. To make cyanine dyes suitable for intracellular labeling, the pendant sulfonic acids would likely need to be replaced with more cell-permeable functionality, e.g., morpholines, sulfonamides, or PEG-like spacers, using methods known in the art.

To maximize both the efficiency of energy transfer and the generality of this approach, an optimal targeting strategy would allow placement of the fluorophore as close as possible to the luciferin binding site, with minimal perturbation to the size of the luciferase reporter (see FIG. 1). Thus, the invention includes modified luciferases that include a HaloTag™ protein sequence appended at the C-terminus or N-terminus of a luciferase.

### Polypeptides, Nucleic Acids, Vectors and Kits

The present invention includes the modified luciferase polypeptides themselves, with and without a protein of interest fused in frame, as well as nucleic acids encoding such polypeptides, optionally operably linked to a regulatory, promoter, enhancer, or silencer sequence, and vectors comprising such nucleic acids. As used herein, a “vector” includes both viral vectors (e.g., recombinant retroviruses, adenovirus, adeno-associated virus, lentivirus, or herpes simplex virus 1) and non-viral vectors (e.g., recombinant bacterial or eukaryotic plasmids). In some embodiments, the vectors are cloning vectors that include a number of restriction enzyme recognition sites that allow a nucleic acid encoding a protein of interest to be inserted such that it will be expressed in frame with the modified luciferase, i.e., as a fusion protein comprising the modified luciferase and the protein of interest. Alternatively or in addition, the vector can include restriction enzyme recognition sites that allow the insertion of one or more regulatory, promoter, enhancer, or silencer sequences operably linked to the modified luciferase.

In addition, the invention includes kits comprising the polypeptides, nucleic acids, and vectors described herein. The kits can also include a BRET-acceptor NIR fluorophore, e.g., as described herein, and instructions for use in methods described herein.

Luciferase-NIR Fluorophore Fusion Proteins

Also described herein are fusion proteins that include luciferase and a protein that fluoresces in the NIR range, e.g., above 600 nm, e.g., above 650 nm. Among the most red-shifted fluorescent proteins presently known are mPlum, which is maximally excited in the red (590 nm) and emits at 649 nm, and mCherry, which is maximally excited in the red (587 nm) and emits at 610 nm (Shaner et al., Nature Biotechnology, 22:1567-1572 (2004)); others include mStrawberry and mRaspberry. See, e.g., U.S. Pat. App. Pub. No. 2006/0099679 to Tsien and Wang. Thus, the invention includes nucleic acids encoding these fusion proteins, vectors including those nucleic acids, and cells and transgenic animals expressing these fusion proteins.

Targeting Fluorophores

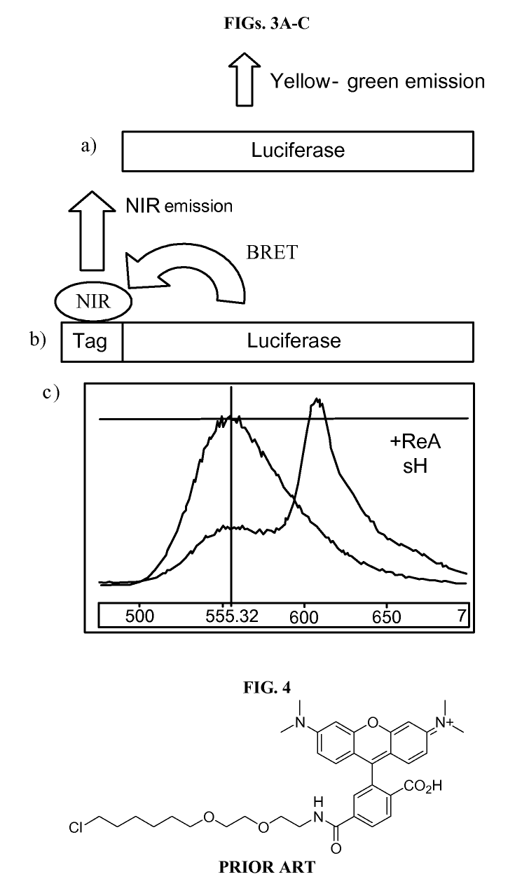

Resonance energy transfer from luciferase to the near-IR requires an acceptor fluorophore that has a strong near-IR emission and can be targeted to within the Forster distance from luciferase for efficient energy transfer. Described herein are targetable, cell-permeable small molecule near-IR fluorophores (sNIRFs) that will shift the output of a luciferase, such as firefly luciferase, to >650 nm (see FIGS. 3A and 3B). In some embodiments, these sNIRFs are bis-arsenicals that bind to tetracysteine-tagged luciferases, and in some embodiments the sNIRFs are analogs of oxazine laser dyes, e.g., analogs of oxazine 170.

The requirements for dyes useful in the targeting strategies described herein include bright fluorescence, cell-permeability, and tight binding to the tetracysteine tag. The widely used water-soluble cyanine near-IR dyes are not cell-permeable and direct attachment of arsenic to the fluorophore would not be as rigidly displayed as with oxazine dyes. On the other hand, like fluorescein, oxazine dyes are well-suited for the rigid display of arsenic atoms or antimony needed to bind to tetracysteine tags, but no bis-arsenical (or bis-Sb) near-IR fluorophores have previously been described.

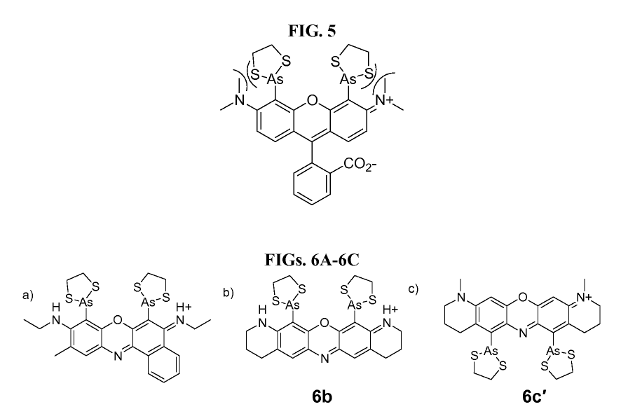

Reported bis-arsenical fluorophores lack alkyl substitution adjacent to the arsenic atoms (FIG. 2). See, e.g., U.S. Pat. App. Pub. No. 2005/0131217 to Tsien and Griffin, and U.S. Pat. Nos. 5,932,474; 6,008,378; 6,054,271; 6,451,569; and 6,686,458, all to Tsien and Griffin. Indeed, bis-arsenical derivatives of the red dyes tetramethylrhodamine and Rhodamine B have been reported to be non-fluorescent, ostensibly due to steric repulsion between the dialkylamino groups and the As(EDT) moieties (FIG. 5) (Adams et al., J. Am. Chem. Soc., 2002, 124:6063-76). On the other hand, an oxazine dye based on Nile Red has been reported to yield a fluorescent bis-arsenical (BArNile, FIG. 2) (Nakanishi et al., Anal. Chem., 2001, 73:2920-28). Nile Red is a solvochromatic dye that is poorly fluorescent in water and thus unsuitable as an acceptor fluorophore for luciferase. However, the fluorescence of this dye as a bis-arsenical in hydrophobic environments supports the idea that it is not amino substitution adjacent to the As(EDT) moiety per se that disrupts fluorescence, but rather steric repulsion by the attached dialkyl groups.

Described herein are novel targetable bis-arsenical near-IR fluorophores and related antimony fluorophores based on near-IR oxazine dyes, including bis-arsenical near-IR-emitting oxazine dyes that have less bulky substituents near the arsenic targeting moieties, or arsenic atoms on more distal locations on the fluorophore (FIGS. 6A-B), which is expected to address any steric hindrance that may be preventing fluorescence of bis-arsenical rhodamine dyes. The invention includes oxazine-based near-IR fluorophores that can be targeted to a tetracysteine tag, wherein arsenic atoms are incorporated into near-IR oxazine dyes following the paradigm of FlAsH and ReAsH (see FIGS. 6A-B).

The laser dye oxazine 170 has the features needed to function as an acceptor dye for BRET from luciferase to the near-IR: cell-permeability, high extinction coefficient and quantum yield in water, and significant emission in the near-IR. The present inventors postulated that bis-arsenical oxazine 170 (As₂Ox170, FIG. 6a) would yield a fluorescent dye, in contrast to tetramethylrhodamine and Rhodamine B, due to decreased steric repulsion between the mono-substituted amino groups and the As(EDT) moiety. Bearing out this prediction, as described herein, As₂Ox170 is initially non-fluorescent, but forms a deep red fluorescence after standing on a TLC plate, as is observed for FlAsH and ReAsH (Adams et al., J. Am. Chem. Soc., 2002, 124:6063-76). Like FlAsH and ReAsH, As₂Ox170 is cell-permeable, and highly fluorescent within cells. Surprisingly, however, As₂Ox170 fails to fluorescently label tetracysteine-tagged proteins. The lack of binding—rather than an inability to fluoresce—was verified by MALDI-MS. Preliminary molecular modeling studies with MM2, a molecular mechanics modeling package (Allinger, J. Am. Chem. Soc. 99:8127-8134 (1977); Allinger et al., J. Comp. Chem. 9:591-595 (1988); Lii et al., J. Comp. Chem. 10:503-513 (1989); available from Tripos, Inc., St Louis, Mo.), as part of the ChemDraw™ 3D Ultra software package (CambridgeSoft) suggested that interaction with the canonical CCPGCC (SEQ ID NO:12) tag is disfavored.

To further explore the possibility of using a bis-arsenical oxazine dye with a CCPGCC (SEQ ID NO:12) tag, a new oxazine dye (AB2, Scheme 1) was synthesized that further reduces the steric hindrance of Oxazine 170, while maintaining similar spectral properties (see Preliminary Data). Bis-arsenical AB2 (FIG. 6B) is expected to have the ability to bind to the canonical tetracysteine tag. Molecular modeling studies with MM2 suggest that this dye, unlike As₂Ox170, will form a favorable complex with a canonical CCPGCC (SEQ ID NO:12) tag.

An alternative method to avoid steric interference with the fluorescence or the binding of bis-arsenicals to tetracysteine tags is simply to place the arsenic atoms at a different location on the fluorophore that avoids the deleterious steric interaction, but maintains the same relative positioning of the arsenics. An example of such a fluorophore is shown in FIG. 6C. However, this compound is not accessible using the mercuration chemistry described for the synthesis of FlAsH and ReAsH. This molecule can be synthesized using halogen-magnesium exchange chemistry (Knochel et al., Angew Chem. Int. Ed. Engl., 2003, 42:4302-20). In addition to allowing the placement of arsenic atoms in different locations on the fluorophore, this chemistry is much more versatile, and can allow the incorporation of metals other than arsenic at each location. Of particular interest is antimony, which is similarly thiophilic, but of lower toxicity.

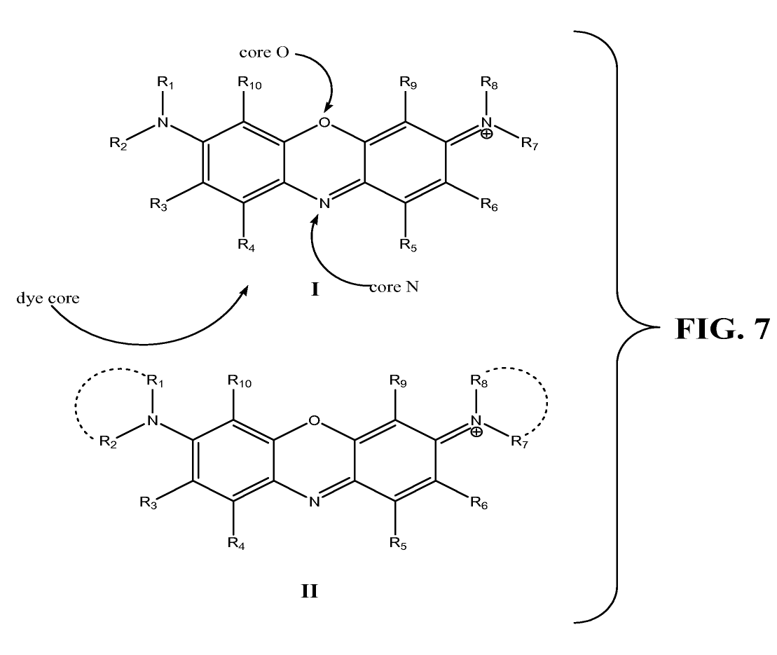

Referring now to FIG. 7, more generally, dyes including cations of Structure (I) are provided that can target a tagged luciferase. Such dyes include a dye core that includes two pairs of functional groups; a first pair on the same side of the dye core as a core oxygen and defined by functional groups R₉ and R₁₀, and a second pair on the same side of the dye core as a core nitrogen (opposite the oxygen) and defined by R₄ and R₅. Each functional group of one of such pairs is an arsenic-containing moiety or an antimony-containing moiety. Each functional group of the second pair of functional groups, along with all other functional groups linked to the dye core, are selected so that the dyes fluoresce at a desired wavelength and sterically permit conjugation of the dye to the tagged luciferase through arsenic or antimony.

When R₄ and R₅ are each an arsenic-containing moiety or an antimony-containing moiety, R₃, R₆, R₉ and R₁₀ can each independently be H, F, Cl, Br, I, OH, or a first moiety that includes up to 12 carbon atoms, and R₁, R₂, R₇ and R₈ can be H or a second moiety that includes up to 12 carbon atoms. In instances when R₄ and R₅ are each an arsenic-containing moiety or an antimony-containing moiety, R₁, R₂, R₃ and R₁₀ and/or R₆, R₇, R₅ and R₉ together with one or more of its immediate neighbors can define one or more ring systems, each including up to 14 carbon atoms. In some instances, it is preferable that R₄ and R₅ be the targeting moieties, which allows R₁ and/or R₂ and R₇ and/or R₅ to be hydrocarbon groups, e.g., alkyl groups, which can red-shift the absorption and/or emission of the corresponding dyes toward longer wavelengths. Alkyl substitution effects are discussed in “LUCIFERINS”, U.S. Provisional Patent Application No. 60/904,731, filed on Mar. 2, 2007, and U.S. patent application Ser. No. ______ [Attorney Docket No. 07917-306001], which is filed concurrently herewith by the same inventor, both of which are incorporated herein by reference in their entirety.

When R₉ and R₁₀ are each an arsenic-containing moiety or an antimony-containing moiety, large groups at any one of positions 1, 2, 7 or 8 can, in some instances, sterically restrict access to the arsenic-containing or antimony-containing moieties. This crowding can, in some instances, prevent targeting of the desired luciferase. To reduce the effects of such crowding, R₁ and R₈ can each be H, R₂ and R₇ can each be H or together with its immediate neighbor R₃ or R₆, respectively, can define one or more ring systems, each including up to 14 carbon atoms, while R₃, R₄, R₅, and R₆ can each be independently H, F, Cl, Br, I, OH, or third moiety that includes up to 12 carbon atoms. R₃ and R₄ and/or R₅ and R₆ together with one or more of its immediate neighbors can also define one or more ring systems, each including up to 14 carbon atoms. Confining one or more of R₂, R₃, R₄, R₅, R₆ or R₇ in a ring system can reduce steric crowding in the vicinity of the arsenic-containing or antimony-containing moieties, allowing these targeting moieties to target a selected tagged luciferase.

The first, second or third moiety including up to 12 carbon atoms can also include, e.g., one or more of N, O, P, S, F, Cl, Br, or I. For example, N can be part of an amino group, an amide group or an imine group. For example, O can be part of hydroxyl group, a carboxylic acid group, an ester group, an anhydride group, an aldehyde group, a ketone group or an ether group. For example, S can be part of a thio-ester group, a thiol group or a thio-ether group. For example, P can be part of a phosphate group, a phosphonate group, a phosphine group, or a phosphoramide group.

For example, the first, second or third moiety including up to 12 carbon atoms can be or can include a hydrocarbon fragment, e.g., an alkyl group, an alkenyl group, an alkynyl or an aryl group, or a hydrocarbon fragment that is substituted with one or more of N, O, P, S, F, Cl, Br, or I.

Any ring system defined herein can further include in a ring or substituted on the ring, e.g., one or more of N, O, P, S, F, Cl, Br, or I. For example, the balance of the 14 carbons atoms not in a ring can substitute a ring, e.g., in the form of hydrocarbon fragments, e.g., an alkyl group, an alkenyl group, an alkynyl or an aryl group, or a hydrocarbon fragment that is substituted with one or more of N, O, P, S, F, Cl, Br, or I. For example, N can be part of an amino group, an amide group or an imine group. For example, O can be part of hydroxyl group, a carboxylic acid group, an ester group, an anhydride group, an aldehyde group, a ketone group or a ether group. For example, S can be part of a thio-ester group, a thiol group or a thio-ether group.

In some preferred embodiments, the one or more ring systems define one or more 5, 6, and/or 7-membered rings.

In some embodiments, when R₄ and R₅ are each an arsenic-containing moiety or an antimony-containing moiety, R₁ and R₂ and R₇ and R₈ can together define a ring such that the compounds are represented by Structure (II) of FIG. 7.

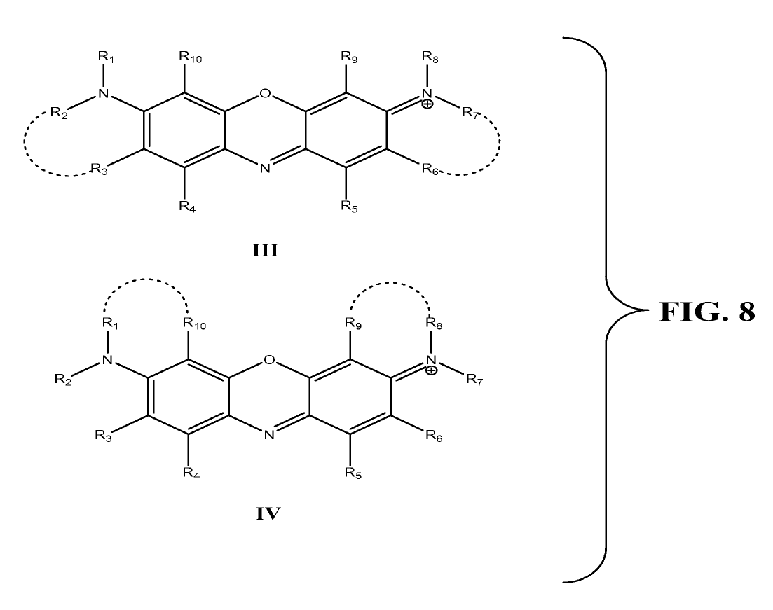

In certain embodiments, when R₄ and R₅ or R₉ and R₁₀ are each an arsenic-containing moiety or an antimony-containing moiety, R₂ and R₃ and R₆ and R₇ together define a ring such that the compounds are represented by Structure (III) of FIG. 8.

In certain embodiments, when each R₄ and R₅ are each an arsenic-containing moiety or an antimony-containing moiety, R₁ and R₁₀ and R₈ and R₉ together define a ring such that the compounds are represented by Structure (IV) of FIG. 8.

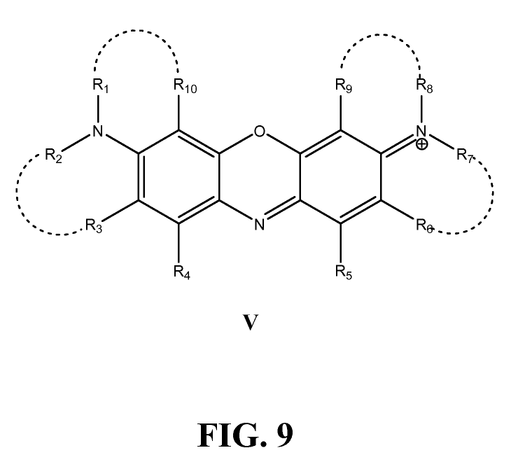

In some preferred embodiments, when R₄ and R₅ are each an arsenic-containing moiety or an antimony-containing moiety, R₁ and R₁₀, R₂ and R₃, R₈ and R₉ and R₆ and R₇ together define a ring such that the compounds are represented by Structure (V) of FIG. 9. This configuration effectively “ties back” core functional groups, allowing for good access of tagged moieties to the targeting moieties.

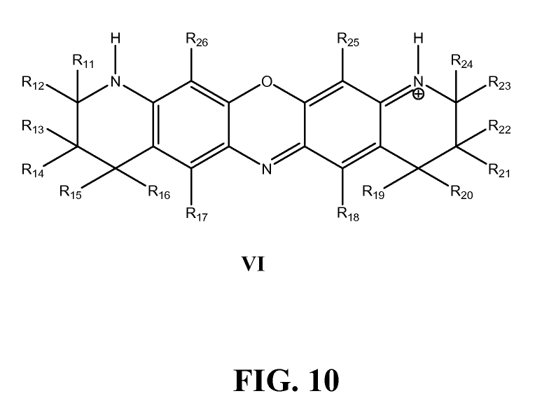

In other preferred embodiments, cations are represented by Structure (VI) of FIG. 10, in which either R₁₇ and R₁₉ or each R₂₅ and R₂₆ are each an arsenic-containing moiety or an antimony-containing moiety. When R₁₇ and R₁₉ are each an arsenic-containing moiety or an antimony-containing moiety, R₁₁, R₁₂, R₁₃, R₁₄, R₁₅, R₁₆, R₁₉, R₂₀, R₂₁, R₂₂, R₂₃, R₂₄, R₂₅ and R₂₆ can each be independently H, or a first moiety that includes up to 8 carbon atoms. When R₂₅ and R₂₆ are each an arsenic-containing moiety or an antimony-containing moiety, R₁₁, R₁₂, R₁₃, R₁₄, R₁₅, R₁₆, R₁₇, R₁₈, R₁₉, R₂₀, R₂₁, R₂₂, R₂₃ and R₂₄ can each be independently H, or a second moiety that includes up to 8 carbon atoms.

The first or second moiety including up to 8 carbon atoms can also include, e.g., one or more of N, O, P, S, F, Cl, Br, or I. For example, N can be part of an amino group, an amide group or an imine group. For example, O can be part of hydroxyl group, a carboxylic acid group, an ester group, an anhydride group, an aldehyde group, a ketone group or an ether group. For example, S can be part of a thio-ester group, a thiol group or a thio-ether group. For example, P can be part of a phosphate group, a phosphonate group, a phosphine group, or a phosphoramide group.

For example, the first or second moiety including up to 8 carbon atoms can be or can include a hydrocarbon fragment, e.g., an alkyl group, an alkenyl group, an alkynyl or an aryl group, or a hydrocarbon fragment that is substituted with one or more of N, O, P, S, F, Cl, Br, or I.

Any dye described herein can include, e.g., F⁻, Cl⁻, Br⁻, I⁻, ClO₄⁻, CH₃COO⁻, BF₄⁻ or PF₆⁻ as a counterion. This list is not intended to be exhaustive, as other anions are available.

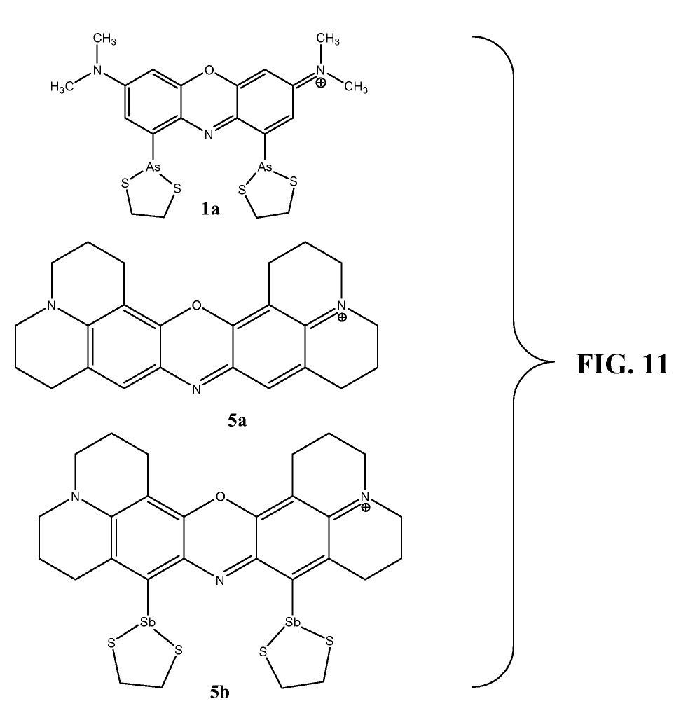

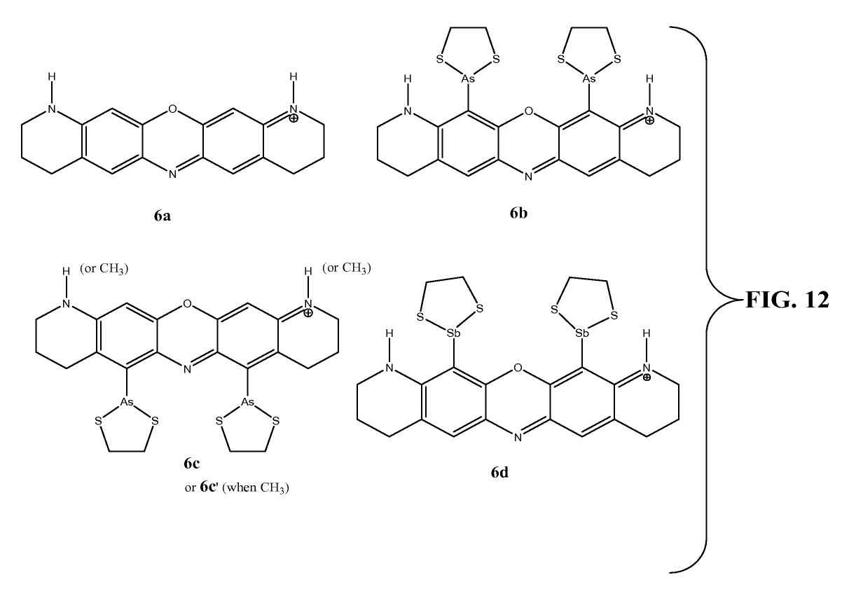

Specific examples of dye cations are shown in FIGS. 11 and 12. For example and by reference particularly to FIG. 11, the dye can include a cation of Structure (1a) or Structure (5b). As will be described below, dyes that include cations of Structure (5b) can be made, e.g., from compounds that include cations of Structure (5a). For example and by reference particularly to FIG. 12, the dye can include a cation of Structure (6b), Structure (6c), Structure (6c′) or Structure (6d), all of which can be made, e.g., from compounds that include cations of Structure (6a), as will be further described below.

Methods of Making Targeting Fluorophores

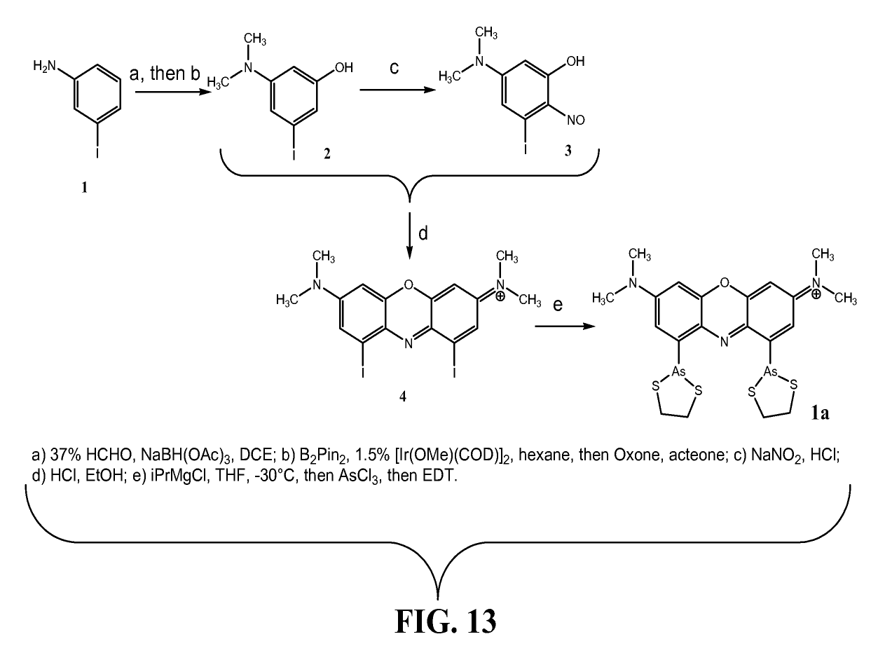

Referring now to FIG. 13, dyes that include cations of Structure (1a) can be made by di-methylating 3-iodoaniline (1) using (a) HCHO, NaBH(OAC)₃ and DCE, and then treating the resulting di-methylated compound with (b) (B₂Pin₂) and [Ir(OMe)(COD)]₂ in hexane, followed by Oxone in acetone, to liberate the hydroxylated di-methylamino compound (2). Compound (2) can be converted to nitroso compound (3) by treatment of compound (2) with (c) NaNO₂ and HCl. Compounds (2) and (3) can be coupled using (d) HCl in ethanol, liberating a compound that includes cation (4). Cation (1a) can be prepared from the compound that includes cation (4) by treatment with (e) isopropylmagnesium chloride in THF at −30° C., followed by treatment with AsCl₃ and then EDT.

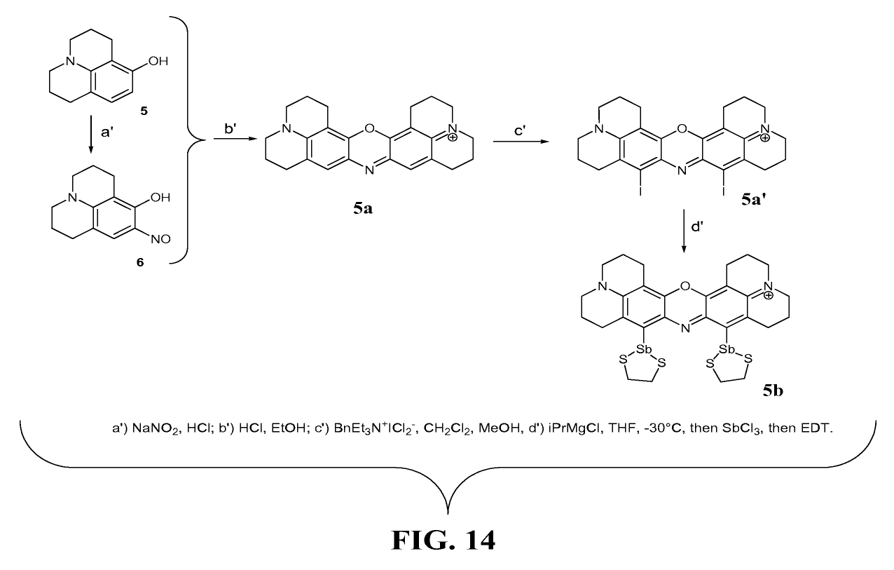

Referring now to FIG. 14, compounds that include cations of Structure (5b) can be prepared from precursor compounds that include cations of Structure (5a). Cations of Structure (5a) can be prepared by treating compound (5) with (a′) NaNO₂ in HCl to provide nitroso compound (6), which can be coupled by treatment with (b′) HCl in ethanol to liberate compounds that include cations of Structure (5a). Treatment of compounds that include cations of Structure (5a) with (c′) BnEt₃N⁺ICl₂⁻ in methylene chloride and ethanol provides corresponding iodo compounds that include cations of Structure (5a′). Finally, treatment of compounds that include cations of Structure (5a′) with (d′) isopropylmagnesium chloride in THF at −30° C., followed by treatment with SbCl₃ and then EDT gives the desired compounds that include cations of Structure (5b).

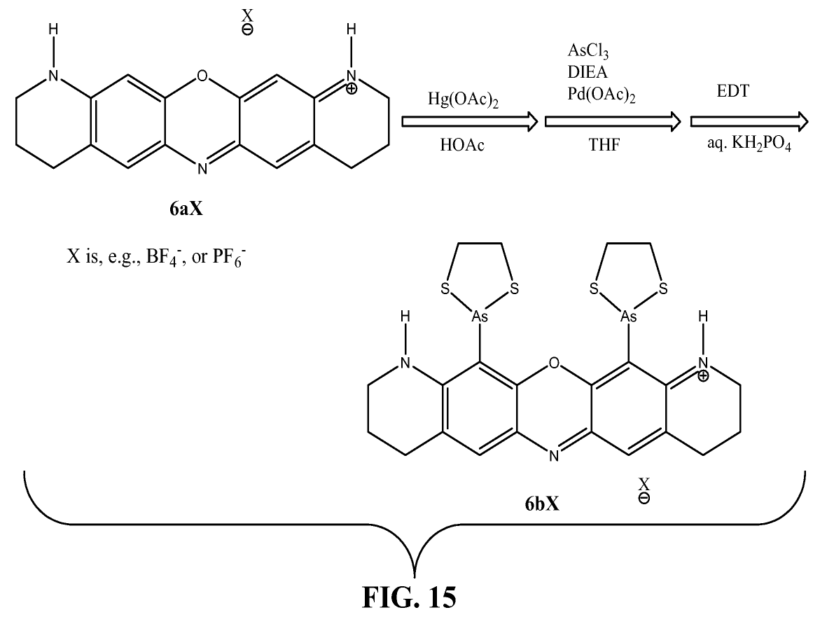

Referring now to FIG. 15, targeting dyes of Structure (6bX) in which X⁻ is, e.g., BF₄⁻ or PF₆⁻, can be made by treating precursor compounds of Structure (6aX) with (1) Hg(OAc)₂ in acetic acid to produce the novel di-mercurated compounds in which mercury is bonded to the dye core beta to oxygen; and then treatment of the mercurated compounds with (2) AsCl₃, DIEA, and Pd(OAc)₂ in THF, followed by (3) EDT in aqueous KH₂PO₄ to give desired compounds of Structure (6bX).

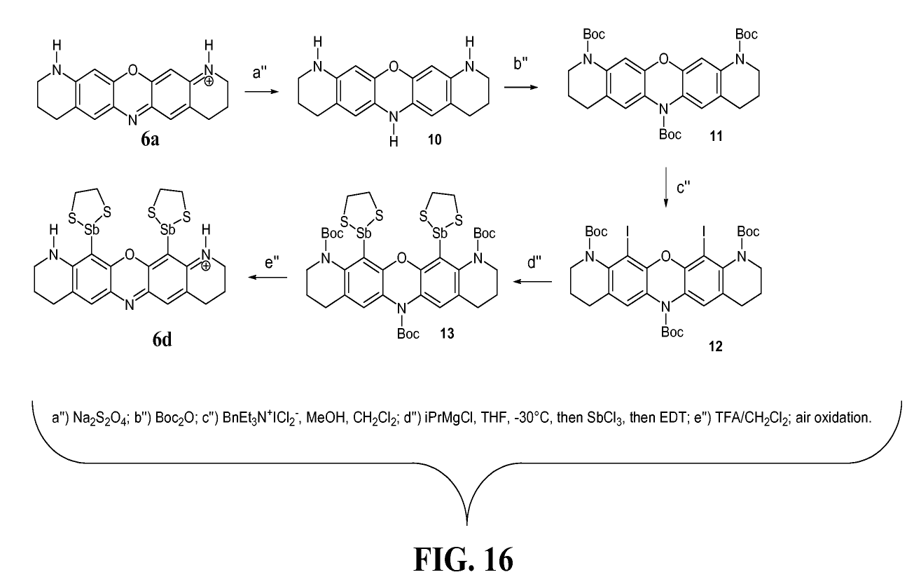

Referring now to FIG. 16, targeting dyes that include cations of Structure (6d) can be made by treating precursor compounds that include cations of Structure (6a) with (a″) Na₂S₂O₄ to liberate reduced compound (10), followed by treatment of compound (10) with (b″) Boc₂O to give protected compound (11). Protected compound (11) is then treated with (c″) BnEt₃N⁺ICl₂⁻ in methylene chloride and methanol to provide the corresponding iodo compounds (12) in which two iodine atoms are beta to the oxygen on the core. Treatment of compound (12) with (d″) isopropylmagnesium chloride in THF at −30° C., followed by treatment with SbCl₃ and then EDT gives compound (13). Finally, deprotection with (e″) TFA in methylene chloride, followed by air oxidation gives the desired targeting dyes that include cations of Structure (6d).

Cell-Permeable Near-IR Dyes that Bind HaloTag™ Protein

The near-IR oxazine dyes described herein can be readily attached to a chloroalkyl group (see FIG. 7), allowing targeting of the fluorophore to a HaloTag™ protein. The chloroalkyl group can be synthesized as reported (see U.S. Pat. Pub. No. 2005/0272114).

Simple amide bond formation, can be used to attach the respective fluorophore. A wide variety of synthesized or commercially-available dyes can also be utilized. These targetable fluorophores can be used to shift the emission of HaloTag™ protein-luciferase fusions to the near-IR.

Methods of Use

The methods and compositions described herein can be used for in vivo imaging because they will improve the speed, detection limit, and depth penetration of bioluminescence imaging. For example, the present methods can be used for the rapid and inexpensive evaluation of disease progression and response to potential therapeutics in small animals. Diseases that can be evaluated include cancers, infectious diseases (e.g., by monitoring NF-kappaB activation), and autoimmune diseases. The methods can also be used for monitoring inhibition of enzymatic activity by a drug candidate, to evaluate the efficacy of the drug candidate

In general, the methods will be performed on cells or animals (e.g., non-human mammals, e.g., experimental animals) that express a mutated luciferase that includes one or both of a tetracysteine tag or a HaloTag™ protein, alone or in addition to AGT (SNAP-tag, see Tirat et al., Int. J. Biol. Macromol., 39:66-76 (2006), Epub 2006 Feb. 28), dihydrofolate reductase (DHFR), FK506-binding protein (FKBP12), HisTag (e.g., His₆ Tag), e.g., a luciferase reporter construct. Sufficient amounts of luciferin and an appropriate fluorophore are then added or administered to the cells or animals, and images of the NIR bioluminescence obtained. When an experimental animal is used, the cells containing the NIR bioluminescence can be identified and excised, e.g., using the native fluorescence of the targeted sNIRF, and evaluated further, e.g., using assays for gene expression, protein expression, or other genetic or biochemical parameters.

Imaging Methods

The methods described herein can be practiced with any imaging system that can detect near infrared bioluminescence, e.g., the in vivo imaging systems described in Doyle et al., Cellular Microbiology, 6(4):303-317 (2004). A commonly used system is the Xenogen IVIS™ Imaging System (Xenogen Corp., Hopkinton, Mass.), but systems from Hamamatsu Photonics (e.g., the AEQUORIA™ system, Roper Scientific Instrumentation (Trenton, N.J.)), and Kodak could also be used. See, e.g., U.S. Pat. Pub. No. 2004/0021771 and 2005/0028482.

Depending on the strength of the bioluminescence, and the location and size of the structure desired to be imaged, bioluminescence that is up to several millimeters from the surface can be detected with planar reflectance imaging (see, e.g., Ntziachristos et al., Nat. Biotechnol., 23(3):313-320). Deeper tissues can be imaged using tomographic imaging methods, e.g., tomographic bioluminescence imaging methods, see, e.g., Chaudhari et al., Phys. Med. Biol., 50(23):5421-41 (2005); Dehghani et al., Opt. Lett., 31(3):365-7 (2006)).

The methods can be used to image expression of any reporter construct including the modified luciferase described herein, i.e., a luciferase including a tetracysteine tag or a HaloTag™ protein. The reporter construct can also include a gene of interest, and can be integrated into the genome of a cell or non-human animal or can be independently replicating, e.g., on a plasmid vector. The cells can express the construct stably or transiently. The imaging can be performed, for example, in cells transiently expressing a modified luciferase reporter construct, e.g., cells transfected with a plasmid or infected with a virus; any of a number of methods known in the art for inducing gene expression in a cell can be used. Alternatively, the imaging can be performed in cells stably expressing a modified luciferase reporter construct, e.g., cells including in their genome at least one copy of a modified luciferase reporter construct. Finally, the imaging can be performed in animals, e.g., living animals, e.g., transgenic non-human mammals that express in their somatic and/or germ cells a modified luciferase reporter construct as described herein, as well as tissues from those animals.

Once cells, animals, or tissue expressing a modified luciferase reporter construct as described herein have been obtained, the methods include contacting the cells, tissue, or animals with a NIR dye as described herein that is appropriate for the modified luciferase used. For example, if the modified luciferase includes a tetracysteine tag, the NIR dye will be one that binds to the tetracysteine tag, e.g., a bis-arsenical dye as described herein. General methods for labeling tetracysteine-tagged proteins with bis-arsenical dyes are described in U.S. Pat. App. Pub. No. 2005/239135, to Bogoev et al., If the modified luciferase includes a HaloTag™ protein, the NIR dye will be one that binds to the HaloTag™ protein, e.g., a chloroalkyl-tethered fluorophore.

Bis-arsenicals can freely cross membranes, and are expected to distribute throughout the body. While the toxicity of bis-arsenical fluorophores such as ReAsH is currently unknown, there is data for similar molecules. In particular, derivatives of the trypanosomiasis drug, melarsonyl, contain the same arsenic-EDT complex found in the bis-arsenical fluorophores FlAsH and ReAsH. These derivatives show no apparent signs of acute toxicity at concentrations<100 μmol/kg in mice (Loiseau et al., Antimic. Agents and Chemo., 2000, 44:2954-61). For reference, the cancer drug methotrexate has a two-fold lower LD₅₀ than melarsonyl. Other related arsenic-containing organic compounds, such as arsanilic acid, are much less toxic, with an LD₅₀ in mice of 248 mg/kg.

It is important to note that the amount of bis-arsenical needed for labeling is much lower than the therapeutic dose of melarsonyl. The affinity between FlAsH and the tetracysteine tag is very high, with a dissociation constant of about 4 pM (Adams et al., J. Am. Chem. Soc., 2002, 124:6063-76). Given the affinity of bis-arsenicals for the tetracysteine tag, the concentration needed to label a tetracysteine-tagged protein in the mouse is estimated to be 100-1000-fold less than a toxic dose of melarsonyl. To achieve a 1 μM concentration in a 10 g mouse (1 μM is a typical concentration for labeling tissue culture cells) would likely require about 1-10 nmol of substrate (depending on the effective volume of the mouse). This works out to 0.1-1 μmol/kg, or 100-1000-fold less than the toxic dose of melarsonyl. Thus, bis-arsenical fluorophores are expected to be well-tolerated by mice at the levels needed to label TC-tagged luciferase. One of skill in the art will appreciate that the amount of dye administered will depend on whether a cell, tissue, or animal is used. In general, an amount sufficient to produce a detectable NIR signal is used.

Transgenic Animals

The present invention also includes non-human transgenic animals, e.g., transgenic rodents, expressing a modified luciferase in their somatic and/or germ cells. Methods for generating non-human modified luciferase transgenic animals are known in the art. Such methods typically involve introducing a nucleic acid, e.g., a nucleic acid encoding a modified luciferase, into the germ line of a non-human animal to make a transgenic animal. Exemplary modified luciferase sequences are described herein. Although rodents, e.g., rats, mice, rabbits and guinea pigs, are typically used, other non-human animals can be used. In these methods, typically one or several copies of the nucleic acid are incorporated into the DNA of a mammalian embryo by known transgenic techniques (see, e.g., Nagy et al., Manipulating the Mouse Embryo: A Laboratory Manual, 3^{rd} Ed., Cold Spring Harbor Laboratory Press (2003)). A protocol for the production of a transgenic rat can be found in Bader et al., Clin. Exp. Pharmacol. Physiol. Suppl., 3:S81-S87 (1996).

Such methods can also involve the use of tissue-specific promoters to generate tissue-specific transgenic animals, for example, a pancreatic beta cell-specific transgenic animal can be created using a modified luciferase linked to a diabetes-related gene driven by an insulin promoter.

Transfected or Knockout Cell Lines

Included in the present invention are cells that stably or transiently express a modified luciferase, e.g., isolated host cells. Any of a number of methods known in the art for creating cells that stably or transiently express a modified luciferase can be used to make these cells. See, e.g., Freshney, Culture of Animal Cells: A Manual of Basic Technique (Wiley-Liss; 5th edition (2005)); and Sambrook and Russell, Molecular Cloning: A Laboratory Manual (Cold Spring Harbor Laboratory Press; 3rd edition (2001)). For example, genetically engineered cells can be obtained using known methods, e.g., from a prokaryotic or eukaryotic cell, e.g., an embryonic stem cell or other mammalian cell, e.g., a primary or cultured cell (e.g., a cell in a cell line), in which a modified luciferase has been introduced. A modified luciferase nucleic acid, or a vector including the modified luciferase nucleic acid as described herein, can be introduced into a cell, e.g., a prokaryotic or eukaryotic cell, via conventional transformation or transfection techniques, e.g., calcium phosphate or calcium chloride co-precipitation, DEAE-dextran-mediated transfection, lipofection, electroporation or viral infection. Suitable vectors, cells, methods for transforming or transfecting host cells and methods for cloning the nucleic acid of interest into a vector are known in the art and can be found in, e.g., Sambrook et al., Molecular Cloning: A Laboratory Manual, 3^{rd} Ed., Cold Spring Harbor Laboratory Press (2001).

Cells expressing a modified luciferase can also be injected into an animal, e.g., a non-human mammal, and imaged in vivo. For example, cells of a tumor cell line that express, e.g., stably express, a modified luciferase reporter construct, i.e., a modified luciferase linked to a gene of interest, e.g., an oncogene, can be injected into an animal, e.g., a non-human animal, and allowed to form a tumor. A candidate treatment for cancer, e.g., the type of cancer that the cells were made from, can then be administered to the animal, and gene expression monitored in the cells using a modified luciferase and the imaging methods described herein.

In another embodiment, genetically modified bacteria or viruses that express a modified luciferase as described herein can also be used. The modified bacteria or virus are introduced into an experimental animal or person, and the course of infection can then be followed using the bioluminescence imaging methods described herein.

### EXAMPLES

The invention is further described in the following examples, which do not limit the scope of the invention described in the claims.

### Example 1

### Resonance Energy Transfer from Firefly Luciferase to ReAsH

Firefly luciferase fusion proteins with an optimized tetracysteine (TC) tag (FLNCCPGCCMEP; SEQ ID NO:1) (Martin et al., Nature Biotechnology, 2005, 23:1308-14) fused onto either the amino (TCLuc1) or carboxy (TCLuc2) terminus of the luciferase protein (Promega's pGL3 (GenBank No. U47295.2)) were cloned and expressed (see list in Table 1). The proteins were expressed in E. Coli as GST fusions, purified by glutathione agarose affinity chromatography, and eluted as the TC-tagged protein by cleavage with PreScission™ protease. Emission of TCLuc1 and TCLuc2 was centered at 560 nm, and the emission intensity and peak shape was identical to untagged luciferase, demonstrating that the TC tag did not affect the light output of luciferase.

Treatment of TCLuc1 or TCLuc2 with ReAsH resulted in a dramatic ˜50 nm shift in the emission wavelength maximum to 608 nm, with a corresponding increase in light emitted in the 600-700 nm range (FIG. 3). The efficiency of BRET was significantly better with the N-terminal TC tag (about 80% vs. about 50% apparent, Table 1). The emission spectrum is sharpened, such that the maximum at 608 nm is actually higher than the 560 nm peak of luciferase (FIG. 3).

To help evaluate the role of distance and orientation on the BRET efficiency, additional N-terminal fusions of the TC tag were constructed (Table 1). Inclusion of a flexible (Gly-Ser)₃ repeat spacer between the N-terminal TC tag and the luciferase (TCLuc3) caused a slight lowering of BRET efficiency to about 50%, while truncation of the 5-7 amino acids between the TC tag and the amino terminus of luciferase led to quenching of luciferase emission (TCLuc4) or complete loss of luciferase activity (TCLuc5). The best-performing TCLuc1 fusion was cloned into pcDNA3.1 for expression in mammalian tissue culture cells. This construct lacks a C-terminal peroxisomal targeting sequence, and thus the expressed luciferase will localize to the cytoplasm.

TABLE 1

BRET from tetracysteine-tagged firefly luciferase
constructs to ReAsH.

| SEQ | | | |
|---|---|---|---|
| ID | Tagged | | |
| NO. | construct: | Sequence | BRET: |
| 2 | TCLuc1 | GPLGSFLN [symbol] MEPGS-[LUC] | ~80% |
| 3 | TCLuc2 | GPLGS-[LUC]-FLN [symbol] MEP | ~50% |
| 4 | TCLuc3 | GPLGSFLN [symbol] MEPGSGSGSGS-[LUC] | ~50% |
| 5 | TCLuc4 | GPLGSFLN [symbol] -[LUC] | quenches |
| 6 | TCLuc5 | GPLGSFLN [symbol] -[Δ2-LUC] | inactive |
| 7 | TCLuc6 | GPLGSFLN [symbol] MEP-[LUC] | N/A |
| 8 | TCLuc7 | GPLGSFLN [symbol] GS-[LUC] | N/A |
| 9 | TCLuc8 | Internal placement of [symbol] (35-40) | N/A |
| 10 | TCLuc9 | GPLGSFLN [symbol] MEPGS-[LUC]- | N/A |
| | | FLN [symbol] MEP | |

### Example 2

### Synthesis of Bis-Arsenical Dyes

A bis-arsenical near-IR fluorophore was synthesized based on a commercially available oxazine laser dye, oxazine 170. Mercuration with HgO/TFA followed by transmetallation of the resulting mercurated dye with arsenic was performed as reported (Adams et al., J. Am. Chem. Soc., 2002, 124:6063-76), but with THF instead of NMP as solvent. This allowed the successful synthesis of bis-arsenical oxazine 170 (As₂Ox170, FIG. 6A). These modified conditions substantially improve the yields of bis-arsenical fluorophores, allowing synthesis of FlAsH in nearly twice the yield reported in the literature.

When spotted on a TLC plate, As₂Ox170 behaves similarly to FlAsH: it is initially non-fluorescent, but becomes brightly fluorescent over time. Mammalian tissue culture cells (HeLa) treated with FlAsH or ReAsH show bright intracellular staining in the absence of tetracysteine-tagged proteins and thiol competitors. HeLa cells treated with As₂Ox170 similarly show intracellular staining, with a deep red fluorescence that is less prone to photobleaching than either FlAsH or ReAsH. Surprisingly, however, this dye fails to bind to the canonical tetracysteine tag. MALDI-MS of tetracysteine-tagged protein showed binding of FlAsH and ReAsH, but not As₂Ox170.

Operating under the hypothesis that further reducing the steric hindrance of the alkylamino groups will lead to well-behaved bis-arsenical dyes, a novel tetrahydroquinoline-based oxazine dye, AB2, was synthesized (Scheme 1, below). This dye has an excitation/emission wavelength of 625/642 nm, similar to that of oxazine 170. Based on the bis-arsenical dyes FlAsH and ReAsH, it is expected that incorporation of arsenics into the dye will cause a bathochromatic shift of the excitation/emission wavelength to 640/657 nm.

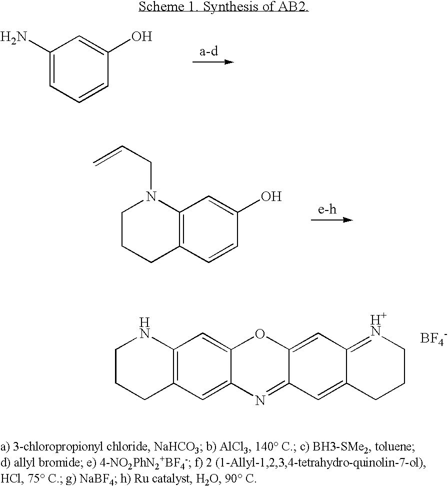

### Example 3

### Targeting of an Acceptor Fluorophore to HaloTag™ Protein-Luciferase Fusion Proteins

A HaloTag™ protein is fused to luciferase and intramolecular BRET to a chloroalkyl-tethered fluorophore is evaluated. An exemplary fluorophore, the commercially-available chloroalkyl-tethered tetramethylrhodamine (Cl-TMR, 555/580) available from Promega Corp (Madison, Wis.), is shown in FIG. 4.

The length of the tether between the HaloTag™ protein and luciferase is varied to optimize both the distance and orientation of the two proteins. BRET efficiency and emission properties of the targetable near-IR dyes are evaluated. Once optimized, the chloroalkyl-tethered near-IR dyes and the corresponding HaloTag™ protein-tagged luciferase constructs are evaluated in mammalian tissue culture cells and in blood.

### Example 4

### Expression and Imaging of Tagged-Luciferase Constructs in Cells and Blood

After in vitro characterization of the emission properties of the purified luciferase fusions (with the TC tag or a HaloTag™ protein), mammalian tissue culture cells transfected with the tagged luciferase constructs are used to evaluate BRET from the luciferase to the acceptor fluorophore in the cellular context. First, the photon counts from untagged and tagged luciferase-expressing cells are compared to determine if the overall light output is unchanged. Luciferase activity, protein expression levels and solubility are evaluated in mammalian cells. Notably, no differences in expression, solubility or activity of the bacterially-expressed tetracysteine-tagged luciferase have been found in vitro.

Next, the ability of bis-arsenical or chloroalkyl dyes to red-shift the light output of cells expressing the tetracysteine-tagged or a HaloTag™ protein-luciferase, respectively, is evaluated. First, using a 600 nm longpass filter, the relative light output above 600 nm is measured for each luciferase construct, with and without addition of the respective fluorophore. As noted above, the addition of ReAsH shifted the emission maximum of tetracysteine-tagged luciferase from 560 to 608 nm in vitro. Notably, the spectral emission of firefly luciferase through about 1 mm of mouse tissue is almost completely attenuated below 600 nm (Rice et al., Journal of Biomedical Optics, 2001, 6:432-440). Second, the emission of the same luciferase constructs is evaluated in blood. This will allow the direct measurement of the light that is not absorbed by hemoglobin or scattered by red and white blood cells, and will provide a good approximation of imaging through animal tissue.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. Compounds comprising cations of Structure (I): 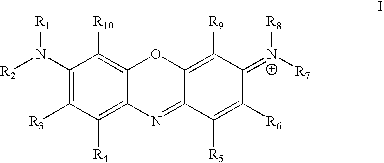
wherein each R₄ and R₅ or each R₉ and R₁₀ is an arsenic-containing moiety or an antimony-containing moiety,
and wherein when R₄ and R₅ are each an arsenic-containing moiety or an antimony-containing moiety, R₃, R₆, R₉, and R₁₀ are each independently H, F, Cl, Br, I, OH, or a first moiety comprising up to 12 carbon atoms; R₁, R₂, R₇ and R₈ are each independently H or a second moiety comprising up to 12 carbon atoms; R₁, R₂, R₃ and/or R₁₀ can define one or more ring systems, each comprising up to 14 carbon atoms; R₆, R₇, R₈ and/or R₉ can define one or more ring systems, each comprising up to 14 carbon atoms,
and wherein when R₉ and R₁₀ are each an arsenic-containing moiety or an antimony-containing moiety, R₁ and R₈ are each H, R₂ and R₇ are each independently H or together with its immediate respective neighbor defines one or more ring systems, each comprising up to 14 carbon atoms, R₃, R₄, R₅ and R₆ are each independently H, F, Cl, Br, I, OH, or third moiety comprising up to 12 carbon atoms, R₃ and R₄ and/or R₅ and R₆ together with one or more of its immediate neighbors may define one or more ring systems, each comprising up to 14 carbon atoms.

2. The compounds of claim 1, wherein R₉ and R₁₀ are each an arsenic-containing moiety or an antimony-containing moiety.

3. The compounds of claim 2, wherein R₄ and R₅ are each H.

4. The compounds of claim 3, wherein R₂ and R₃ together and R₆ and R₇ together each define one or more ring systems, each comprising up to 14 carbon atoms.

5. The compounds of claim 4, wherein each ring system is a 6-membered ring system.

6. The compounds of claim 5, including cations of Structure (6b) or (6d)

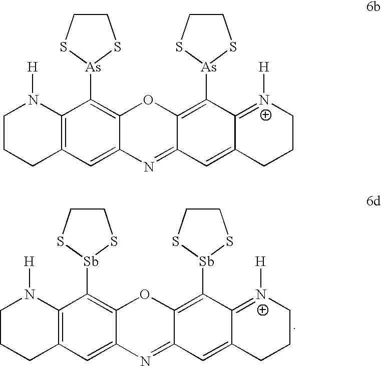

7. The compounds of claim 1, wherein R₄ and R₅ are each an arsenic-containing moiety or an antimony-containing moiety.

8. The compounds of claim 7, including cations of Structure (6c) or (6c′)

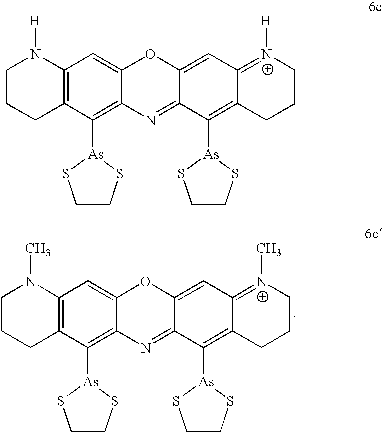

9. The compounds of claim 1, including cations of Structure (1a)

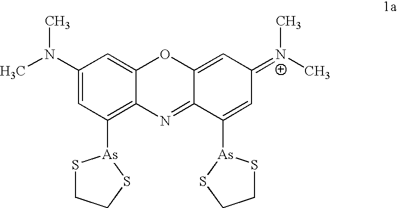

10. The compounds of claim 1, including cations of Structure (5b)

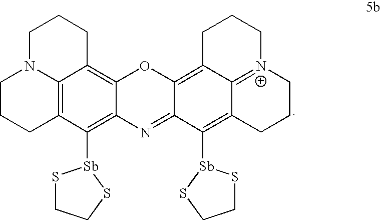

11. A compound comprising cations of Structure (VI): 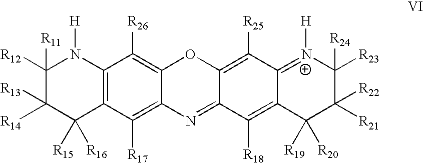
wherein each R₁₇ and R₁₈ or each R₂₅ and R₂₆ is an arsenic-containing moiety or an antimony-containing moiety,
and wherein when R₁₇ and R₁₈ are each an arsenic-containing moiety or an antimony-containing moiety, R₁₁, R₁₂, R₁₃, R₁₄, R₁₅, R₁₆, R₁₉, R₂₀, R₂₁, R₂₂, R₂₃, R₂₄, R₂₅ and R₂₆ are each independently H, or a moiety that includes up to 8 carbon atoms,
and wherein when R₂₅ and R₂₆ are each an arsenic-containing moiety or an antimony-containing moiety, R₁₁, R₁₂, R₁₃, R₁₄, R₁₅, R₁₆, R₁₇, R₁₈, R₁₉, R₂₀, R₂₁, R₂₂, R₂₃ and R₂₄ are each independently H, or a moiety that includes up to 8 carbon atoms.

12. The compounds of claim 11, wherein R₁₇ and R₁₈ are each an arsenic-containing moiety or an antimony-containing moiety and wherein R₁₁, R₁₂, R₁₃, R₁₄, R₁₅, R₁₆, R₁₉, R₂₀, R₂₁, R₂₂, R₂₃, R₂₄, R₂₅, and R₂₆ are each H.

13. The compounds of claim 11, wherein R₂₅ and R₂₆ are each an arsenic-containing moiety or an antimony-containing moiety and wherein R₁₁, R₁₂, R₁₃, R₁₄, R₁₅, R₁₆, R₁₇, R₁₈, R₁₉, R₂₀, R₂₁, R₂₂, R₂₃, and R₂₄ are each H.

14. The compounds of claim 12, wherein the moiety that comprises arsenic or antimony comprises a chelating, sulfur-containing ligand covalently bonded to arsenic or antimony.

15. The compounds of claim 14, wherein the chelating, sulfur-containing ligand covalently bonded to the arsenic or antimony is SCH₂CH₂S.

16. The compounds of claim 1, further comprising an anion selected from the group consisting of ClO₄⁻, BF₄⁻ and PF₆⁻.

17. A conjugate of any compound of claim 1 and a peptide, a polypeptide or a protein.

18. A composition comprising any compound of claim 1.

19. An isolated polypeptide comprising a luciferase polypeptide and at least one tetracysteine tag comprising the sequence CCXXCC (SEQ ID NO:11), wherein the polypeptide is conjugated to any compound of claim 1.

20. The isolated polypeptide of claim 19, wherein the sequence CCXXCC (SEQ ID NO:11) is at the N terminus, the C terminus, or internal to the luciferase polypeptide sequence.

21. An isolated nucleic acid molecule comprising a sequence of nucleotides encoding a modified luciferase polypeptide comprising a luciferase polypeptide and at least one haloalkane dehydrohalogenase mutant.

22. An isolated nucleic acid molecule comprising the nucleic acid molecule of claim 6 in frame with a second sequence of nucleotides encoding a protein, and optionally comprising a third sequence of nucleotides encoding a linker between the modified luciferase and the preselected protein.

23. An isolated nucleic acid molecule comprising the nucleic acid molecule of claim 21 operably linked to a preselected regulatory sequence, enhancer sequence, silencer sequence, or promoter.

24. A host cell comprising the nucleic acid molecule of claim 21.

25. A vector comprising the nucleic acid molecule of claim 21.

26. A host cell comprising the vector of claim 25.

27. An isolated polypeptide comprising a luciferase polypeptide fused in frame with at least haloalkane dehydrohalogenase mutant at one or more of the N terminus and the C terminus.

28. An isolated polypeptide comprising a luciferase polypeptide fused in frame with at least one haloalkane dehydrohalogenase mutant at one or more of the N terminus and the C terminus, wherein the polypeptide is conjugated to any compound of claim 1.

29. An isolated polypeptide comprising the polypeptide of claim 19, fused in frame with a protein of interest.

30. An isolated polypeptide comprising the polypeptide of claim 27, fused in frame with a protein.

31. An isolated polypeptide comprising the polypeptide of claim 28, fused in frame with a protein.

32. A transgenic non-human mammal the nucleated cells of which comprise a transgene encoding the isolated polypeptide of claim 27, wherein the polypeptide is expressed in at least some of the cells of the mammal.

33. The transgenic non-human mammal of claim 32, wherein the mammal is a mouse.

34. A transgenic non-human mammal whose genome is heterozygous for a transgene encoding the isolated polypeptide of claim 27, wherein the polypeptide is expressed in at least some of the cells of the mammal.

35. The transgenic non-human mammal of claim 34, wherein the mammal is a mouse.

36. A method of imaging in a living cell, the method comprising:
providing a cell expressing the modified luciferase polypeptide of claim 27;
contacting the cell with luciferin;
contacting the cell with a near-infrared (NIR) acceptor dye that binds to the polypeptide and undergoes intramolecular biofluorescence resonance energy transfer (BRET) with the modified luciferase polypeptide; and
detecting NIR emission from the NIR acceptor dye.

37. The method of claim 36, wherein the NIR acceptor dye is a bis-arsenical dye that fluoresces above 600 nm.

38. A method of imaging in a living cell, the method comprising:
providing a cell expressing the polypeptide of claim 27;
contacting the cell with luciferin;
contacting the cell with a near-infrared (NIR) acceptor dye that binds to the polypeptide and undergoes intramolecular biofluorescence resonance energy transfer (BRET) with the modified luciferase polypeptide; and
detecting NIR emission from the NIR acceptor dye.

39. The method of claim 38, wherein the NIR acceptor dye is a chloroalkyl-tethered fluorophore dye that fluoresces above 600 nm


SEQUENCE LISTING (13 sequences)

SEQ ID NO: 1 - protein, 12 aa, Artificial Sequence
  Synthetically generated peptide
FLNCCPGCCMEP

SEQ ID NO: 2 - protein, 569 aa, Artificial Sequence
  Synthetic construct
GPLGSFLNCCPGCCMEPGSMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 3 - protein, 567 aa, Artificial Sequence
  Synthetic construct
GPLGSMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVFLNCCPGCCMEP

SEQ ID NO: 4 - protein, 575 aa, Artificial Sequence
  Synthetic construct
GPLGSFLNCCPGCCMEPGSGSGSGSMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 5 - protein, 564 aa, Artificial Sequence
  Synthetic construct
GPLGSFLNCCPGCCMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 6 - protein, 562 aa, Artificial Sequence
  Synthetic construct
GPLGSFLNCCPGCCDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 7 - protein, 567 aa, Artificial Sequence
  Synthetic construct
GPLGSFLNCCPGCCMEPMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 8 - protein, 566 aa, Artificial Sequence
  Synthetic construct
GPLGSFLNCCPGCCGSMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 9 - protein, 550 aa, Artificial Sequence
  Synthetic construct
MEDAKNIKKGPAPFYPLEDGTAGEQLHKAMKRYACCPGCCAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 10 - protein, 581 aa, Artificial Sequence
  Synthetic construct
GPLGSFLNCCPGCCMEPGSMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVFLNCCPGCCMEP

SEQ ID NO: 11 - protein, 6 aa, Artificial Sequence
  Synthetically generated peptide
CCXXCC

SEQ ID NO: 12 - protein, 6 aa, Artificial Sequence
  Synthetically generated peptide
CCPGCC

SEQ ID NO: 13 - protein, 6 aa, Artificial Sequence
  Synthetically generated peptide
LVPGTI